(19) United States
(12) Patent Application Publication
(10) Pub. No.: US 2015/0274538 A1
(43) Pub. Date: Oct. 1, 2015

(54) CORE-SHELL SILICA NANOPARTICLES, METHOD FOR MANUFACTURING THE SAME, METHOD FOR MANUFACTURING HOLLOW SILICA NANOPARTICLES THEREFROM, AND HOLLOW SILICA NANOPARTICLES MANUFACTURED THEREBY

(71) Applicant: DIC Corporation, Tokyo (JP)

(72) Inventors: Jianjun Yuan, Sakura-shi (JP); Hiroshi Kinoshita, Sakura-shi (JP)

(73) Assignee: DIC CORPORATION, Tokyo (JP)

(21) Appl. No.: 14/433,938

(22) PCT Filed: Oct. 9, 2013
(86) PCT No.: PCT/JP2013/077474

§ 371 Date: Apr. 7, 2015

(30) Foreign Application Priority Data
Oct. 10, 2012 (JP) 2012-225043
Oct. 12, 2012 (JP) 2012-226950

(51) Int. Cl. C01B 33/12; A61K 9/48; A61K 8/02; B01J 31/06

(52) U.S. Cl. CPC C01B 33/126; B01J 31/069; A61K 9/4816; A61K 8/0245; A61K 8/0279; C01P 2004/64

(57) ABSTRACT

The present invention relates to a monodisperse core-shell silica nanoparticle including a core layer based on a hydrophobic organic segment (a2) portion of a copolymer (A) containing an aliphatic polyamine chain (a1) containing primary amino groups and/or secondary amino groups and the hydrophobic organic segment (a2) and a shell layer composed of a hybrid based on the aliphatic polyamine chain (a1) portion and silica (B), and also relates to a method for manufacturing such a core-shell silica nanoparticle, a method for manufacturing a hollow silica nanoparticle from such a core-shell silica nanoparticle, and a hollow silica nanoparticle manufactured by this method of manufacture.

### TECHNICAL FIELD

The present invention relates to core-shell silica nanoparticles including a core part (core layer) containing an organic component and a shell layer containing silica and an organic component and to a simple method for manufacturing such core-shell silica nanoparticles. The invention also relates to a method for manufacturing hollow silica nanoparticles by removing an organic component from core-shell silica nanoparticles including a hydrophobic organic segment core that are manufactured by the above method of manufacture and to hollow silica nanoparticles manufactured by this method of manufacture.

### BACKGROUND ART

Recently, efforts have been directed toward the research and development of functional nanostructured materials, and research has been conducted in various industrial fields to develop materials such as nanostructured materials, organic-inorganic hybrid materials, and hierarchical materials. In particular, research has been conducted to utilize the complex functions of nanoparticles having a core-shell structure and nanoparticles having a hollow structure.

Nanoparticles having a core-shell structure, for example, core-shell silica nanoparticles including a polymer core, can be used for applications such as drug delivery systems, controlled release cosmetics, diagnostic materials, optical materials, and the formation of hollow materials. Depending on the properties required for different applications, various studies have been conducted on such silica nanoparticles having a core-shell structure, for example, to introduce a functional organic component or to control the size and structure of the particles.

Nanomaterials having a hollow structure, particularly hollow silica nanoparticles including a silica shell, have properties such as low refractive index, low dielectric constant, low thermal conductivity, and low density and are useful as materials such as antireflection materials, low-dielectric-constant materials, thermal insulation materials, and low-density fillers. Such hollow nanoparticles also allow a target material to be encapsulated into and/or gradually released from the pores present therein to provide various functions. For example, efforts have been directed toward the research of drug delivery systems using hollow silica nanoparticles.

Processes for synthesis of core-shell silica nanoparticles including a polymer core can be broadly classified into emulsion polymerization processes and template processes. Emulsion polymerization processes involve polymerizing a hydrophobic monomer in the presence of silica nanoparticles (sol) to deposit the silica nanoparticles on the surface of the resulting polymer particles, thereby forming a silica shell (see, for example, NPL 1). The thus-formed silica shell, which is a layer of silica nanoparticles physically assembled together, is structurally unstable. For example, the shell layer may collapse after the polymer core is removed. Whereas core-shell silica nanoparticles including a polymer core that are synthesized by emulsion polymerization processes can be used as organic-inorganic hybrid coatings and films, they are difficult to use as core-shell nanoparticles.

Template processes involve performing a silica sol-gel reaction on the surface of synthesized polymer nanoparticles to form a silica shell using the particles as a template. Many template processes are based on the StÖber process, which is a common process for manufacturing silica nanoparticles in which silica is precipitated on the surface of polymer latex particles in the presence of ammonia (see, for example, PTLs 1 and 2). These processes, however, are not environmentally friendly or productive since the sol-gel reaction requires high ammonia concentrations. The core-shell silica nanoparticles synthesized according to PTLs 1 and 2 include a silica shell formed on the surface of polymer particles, which has no organic component introduced in the silica matrix thereof. The polymer latex particles used as the template have a particle size of 50 nm or more; therefore, it is difficult to synthesize core-shell silica nanoparticles having a particle size of 50 nm or less.

Recently, efforts have been directed toward the synthesis of nanosilica by mimicking biogenic silica, and studies have been conducted to synthesize silica nanoparticles in an aqueous medium under mild conditions using polyamines as a template. For example, studies have been conducted to synthesize spherical silica in an aqueous medium using materials such as polyamine-containing polypeptides extracted from biogenic silica, synthetic polyallylamines, cationic polymers, and block copolymers (see, for example, PTLs 3 and 4 and NPLs 2 to 6). For example, PTL 3 discloses that core-shell silica nanoparticles including a cationic polymer core and having a particle size of 35 nm can be synthesized by performing a silica sol-gel reaction in the shell layer of diblock copolymer micelles of amino-containing acrylates using the micelles as a template. This method differs from silica precipitation based on the StÖber process in that the silica layer formed using the polyamine micelles as the template is an organic-inorganic hybrid of a silica matrix and acrylate-containing tertiary polyamines introduced therein.

With these methods, however, it is difficult to manufacture core-shell silica nanoparticles with good monodispersity that have a particle size of 30 nm or less and that can be used in a wide range of fields, including transparent resin fillers. The polyamines that have so far been introduced in the silica matrix of the shell layer are only aromatic polyamines (NPL 4) and acrylate-containing tertiary polyamines (PTL 3). The existing technology for synthesis of silica nanoparticles has not been successful in synthesizing ultrafine core-shell silica nanoparticles with uniform particle size that have a particle size of 5 to 30 nm and that include a shell layer having introduced in the silica matrix thereof an aliphatic polyamine containing primary amino groups and/or secondary amino groups.

Processes for synthesis of hollow silica include processes (template processes) that involve forming a silica shell on a core serving as a template, as described above, and then removing the core therefrom and processes using a reaction interface.

The latter processes involve designing a gas-liquid or liquid-liquid interface and precipitating silica on the interface. For example, a method for manufacturing a hollow silica powder is disclosed that involves spraying a mixture of a silica source and a blowing agent and then performing a sol-gel reaction (see, for example, PTL 5). The hollow silica particles manufactured by this method, however, have a particle size of several microns to several hundreds of microns; therefore, it is difficult to synthesize nano-order hollow silica particles.

Template processes, which involve forming a silica shell on the surface of particles of a material other than silica and then selectively removing only the core material to form hollow silica particles, are suitable for formation of hollow silica nanoparticles using nano-sized templates. The core particles serving as the template may be made of an inorganic compound or an organic polymer. For example, methods for manufacturing hollow silica nanoparticles using inorganic templates are disclosed (see, for example, PTLs 6 and 7). These methods involve forming a silica shell on the surface of nanoparticles such as calcium carbonate, zinc oxide, or iron oxide particles and then removing the core by dissolution in an acid. These inorganic templates, however, have a problem in that they are essentially crystalline and are therefore not suitable for synthesizing perfectly spherical hollow silica nanoparticles.

Organic polymer core particles (nanoparticles) are advantageous over inorganic nanoparticles in that the properties such as the shape, size, structure, and chemical composition of the particles can be easily controlled. For example, methods for manufacturing hollow silica particles having a particle size of 100 nm or more using polymer latex nanoparticles are disclosed (see, for example, PTLs 2 and 8 and NPLs 5 and 6). These methods involve performing a sol-gel reaction on the surface of the polymer latex nanoparticles and then removing the core polymer by firing or solvent extraction. Also reported are methods for manufacturing hollow silica nanoparticles having a diameter of 30 nm using block polymer micelles by precipitating silica in the shell layer of the micelles and then removing the polymer by firing (see, for example, NPL 4).

With these methods, however, it is difficult to manufacture fine hollow silica nanoparticles with good monodispersity that have a particle size of 30 nm or less, preferably 20 nm or less, and that can be used in a wide range of fields, including transparent resin fillers. For example, the hollow silica nanoparticles disclosed in NPL 4 are not monodisperse. The steps such as the synthesis of the polymer nanoparticles serving as the template and the sol-gen reaction are also complicated and are not environmentally friendly or productive. The existing technology for synthesis of hollow silica nanoparticles has not been successful in synthesizing ultrafine hollow silica nanoparticles with uniform particle size that have an average particle size of 5 to 30 nm and that can be manufactured by a simple, environmentally compatible process.

### CITATION LIST

### Patent Literature

PTL 1: Japanese Unexamined Patent Application Publication (Translation of PCT Application) No. 2009-504632

PTL 2: Japanese Unexamined Patent Application Publication No. 2011-42527

PTL 3: Japanese Unexamined Patent Application Publication (Translation of PCT Application) No. 2010-502795

PTL 4: Japanese Unexamined Patent Application Publication No. 2006-306711

PTL 5: Japanese Unexamined Patent Application Publication No. 06-091194

PTL 6: Japanese Unexamined Patent Application Publication No. 2005-263550

PTL 7: Japanese Unexamined Patent Application Publication (Translation of PCT Application) No. 2010-030791

PTL 8: Japanese Unexamined Patent Application Publication (Translation of PCT Application) No. 2009-504632 Non Patent Literature

NPL 1: A. Schmide et al., Macromolecules, 2009, 42, 3721.

NPL 2: D. Morse, Nature, 2000, 403, 289.

NPL 3: N. Kroger, et al., Science, 2002, 298, 584

NPL 4: A. Khanal, et al., J. Am. Chem. Soc., 2007, 129, 1534.

NPL 5: J. Yang, et al., Chem. Mater., 2008, 20, 2875.

NPL 6: M. Pi, et al., Colloids and Surfaces B Biointerfaces, 2010, 78, 193.

### SUMMARY OF INVENTION

### Technical Problem

In view of the foregoing background, an object of the present invention is to provide fine core-shell silica nanoparticles including a shell layer composed of silica hybridized with an aliphatic polyamine containing primary amino groups and/or secondary amino groups, particularly fine core-shell silica nanoparticles with good monodispersity that have a particle size of several tens of nanometers or less, and also to provide a simple, efficient method for manufacturing such core-shell silica nanoparticles. Another object of the present invention is to provide a method for manufacturing, from core-shell silica nanoparticles manufactured by the above method of manufacture, ultrafine hollow silica nanoparticles with a uniform particle size distribution that have a particle size of 5 to 30 nm, particularly 5 to 20 nm, by a simple, efficient, environmentally compatible process, and also to provide hollow silica nanoparticles manufactured by this method of manufacture.

### Solution to Problem

After conducting extensive research to achieve the foregoing objects, the inventors have found that aggregates having a core-shell structure can be easily formed by dissolving a copolymer containing an aliphatic polyamine containing primary amino groups and/or secondary amino groups and a hydrophobic organic segment in an aqueous medium, that a sol-gel reaction of a silica source can be selectively occurred in the shell layer of the aggregates using the aggregates as a template functioning as a silica precipitation catalyst to form core-shell silica nanoparticles including a core layer based on the hydrophobic organic segment portion and a shell layer composed of silica hybridized with the aliphatic polyamine portion, and that the copolymer can be easily removed from the core-shell silica nanoparticles to form silica particles having a hollow structure, thus completing the present invention.

Specifically, the present invention provides a monodisperse core-shell silica nanoparticle including a core layer based on a hydrophobic organic segment (a2) portion of a copolymer (A) containing an aliphatic polyamine chain (a1) containing primary amino groups and/or secondary amino groups and the hydrophobic organic segment (a2) and a shell layer composed of a hybrid based on the aliphatic polyamine chain (a1) and silica (B), and also provides a core-shell silica nanoparticle further containing a polysilsesquioxane and methods for manufacturing such core-shell silica nanoparticles.

The present invention further provides a monodisperse hollow silica nanoparticle having an average particle size of 5 to 30 nm and an inner diameter of 1 to 10 nm.

The present invention further provides a monodisperse hollow silica nanoparticle formed by removing the copolymer (A) from the above core-shell silica nanoparticle and having an average particle size of 5 to 30 nm and an inner diameter of 1 to 10 nm, and also provides a hollow silica nanoparticle further containing a polysilsesquioxane and methods for manufacturing such hollow silica nanoparticles.

In the present invention, the term “monodisperse” means that the width of the particle size distribution falls within ±15% from the average particle size.

### Advantageous Effects of Invention

The core-shell silica nanoparticles provided by the present invention, which are formed via designed self-assembly of a copolymer containing an aliphatic polyamine and a hydrophobic organic segment, are ultrafine silica nanoparticles with good monodispersity that preferably have a particle size of 100 nm or less, particularly preferably 5 to 30 nm. Unlike existing fine core-shell silica particles, the core-shell silica nanoparticles according to the present invention include a shell layer having a molecular hybrid structure in which an aliphatic polyamine is homogeneously hybridized with a silica matrix. These core-shell silica nanoparticles have polyamine-derived chemical or physical functions. For example, polyamines, which are strong ligands, can concentrate metal ions in the silica. Polyamines, which are also reductants, can reduce concentrated noble metal ions into metal atoms to form silica-noble metal hybrid nanoparticles. Polyamines, which are also cationic polymers, can provide functions such as antimicrobial and antiviral effects for the nanoparticles. Thus, the core-shell silica nanoparticles according to the present invention are applicable to numerous fields such as drug delivery systems; controlled release cosmetics; diagnostic materials; optical materials; resin fillers; abrasive fillers; carriers for metal ions, nanometals, and metal oxides; catalysts; and antimicrobial agents. The method of manufacture according to the present invention, which involves the use of a reaction scheme mimicking the formation of biogenic silica, allows the production of ultrafine core-shell silica nanoparticles with good monodispersity that have polyamine functions within a short period of time under mild reaction conditions, e.g., at low temperature and neutral pH.

The hollow silica nanoparticles provided by the present invention exhibit the material properties unique to nano-sized silica and have an extremely small particle size. The outer diameter, pore size, and structure of the hollow silica nanoparticles can be controlled depending on, for example, the conditions for the synthesis of the precursor, i.e., the core-shell silica nanoparticles described above. In particular, fine hollow silica nanoparticles with good monodispersity that have an outer diameter of about 10 nm and a pore size of about 3 nm can be manufactured. Hollow silica nanoparticles each including a plurality of pores of uniform size can also be formed. Thus, the hollow silica nanoparticles according to the present invention are useful for various applications, including numerous fields such as antireflection materials, heat insulation materials, low-dielectric-constant materials, drug delivery systems, catalysts, and cosmetics. The method of manufacture according to the present invention facilitates formation of such hollow silica nanoparticles and allows structural design depending on different applications. In particular, the method of manufacture according to the present invention, in which copolymer aggregates and precursor core-shell silica nanoparticles can be formed within a short period of time under mild conditions, e.g., in water at neutral pH, is environmentally friendly and requires only a simple production process; therefore, it is suited for industrial production.

### BRIEF DESCRIPTION OF DRAWINGS

FIG. 1 is a transmission electron micrograph of spherical core-shell silica nanoparticles obtained in Example 1.

FIG. 2 is a transmission electron micrograph of string-shaped core-shell silica nanoparticles obtained in Example 5.

FIG. 3 is a transmission electron micrograph of multicore core-shell silica nanoparticles obtained in Example 8.

FIG. 4 is a transmission electron micrograph of multicore core-shell silica nanoparticles obtained in Example 11.

FIG. 5 is a transmission electron micrograph of multicore core-shell silica nanoparticles obtained in Example 12.

FIG. 6 is a transmission electron micrograph of core-shell silica nanoparticles obtained in Example 17.

FIG. 7 is a transmission electron micrograph of hollow silica nanoparticles obtained in Example 19.

FIG. 8 shows isothermal curves of the hollow silica nanoparticles obtained in Example 19 for nitrogen gas adsorption (lower) and desorption (upper).

FIG. 9 shows a pore volume distribution curve of the hollow silica nanoparticles obtained in Example 19.

FIG. 10 is a transmission electron micrograph of multipore hollow silica nanoparticles obtained in Example 21.

FIG. 11 shows isothermal curves of the multipore hollow silica nanoparticles obtained in Example 21 for nitrogen gas adsorption (lower) and desorption (upper).

FIG. 12 shows a pore volume distribution curve of the multipore hollow silica nanoparticles obtained in Example 21.

FIG. 13 is a transmission electron micrograph of string-shaped hollow silica nanoparticles obtained in Example 22.

### DESCRIPTION OF EMBODIMENTS

The synthesis of silica (silicon oxide) of designed nanostructure or shape through a sol-gel reaction in the presence of water is believed to require the following three important conditions: (1) a template for inducing the shape or structure, (2) a scaffold for the sol-gel reaction to occur, and (3) a catalyst for hydrolysis and polymerization of a silica source.

To meet the above three conditions for the synthesis of core-shell silica nanoparticles, the present invention uses a copolymer (A) containing an aliphatic polyamine chain (a1) containing primary amino groups and/or secondary amino groups and a hydrophobic organic segment (a2). The copolymer (A), when dissolved into an aqueous medium, can easily form aggregates via molecular self-assembly. These aggregates have a core-shell structure including a core composed of the hydrophobic organic segment (a2) and a shell composed mainly of the polyamine chain (a1).

Using the thus-formed aggregates having a core-shell structure as a template, a sol-gel reaction of a silica source can be selectively performed in the shell layer of the aggregates in a solvent under the catalytic effect of the aliphatic polyamine chain (a1) to form a silica matrix hybridized with the aliphatic polyamine chain (a1). This allows the manufacture of ultrafine core-shell silica nanoparticles with good monodispersity. The present invention is based on these findings.

The present invention also uses the core-shell silica nanoparticles as a precursor required for forming hollow silica nanoparticles. The removal of the copolymer (A) eliminates organic components while retaining the shape of the shell layer, thus forming a hollow structure, and therefore, hollow silica nanoparticles.

The hollow silica nanoparticles manufactured by the above method of manufacture preferably have an average particle size (outer diameter) of 5 to 100 nm, more preferably 5 to 30 nm, even more preferably 5 to 20 nm, particularly preferably 5 to less than 20 nm, most preferably 5 to 15 nm, and has an inner diameter of about 1 to about 30 nm, preferably 1 to 10 nm. These hollow silica nanoparticles have good monodispersity. Specifically, the width of the particle size distribution can be controlled to within ±15% from the average particle size. Hollow silica nanoparticles each including a plurality of pores and string-shaped silica nanoparticles can also be synthesized. Such ultrafine hollow silica nanoparticles readily exhibit the material properties unique to nano-sized particles and also allow various functional materials to be encapsulated into the nanometer-order pores present therein.

By “good monodispersity”, it is meant that the silica particles, whether they are solid or hollow, are nanoparticles having a narrow particle size distribution, i.e., particles larger than the target average particle size and/or particles smaller than the target average particle size are present in smaller proportions.

This provides, for example, the technical advantage of reducing the likelihood of problems due to the presence of large or small particles in larger proportions.

Specifically, for example, the presence of large particles in larger proportions is undesirable because they may hinder the formation of the optimum packing structure and thus result in insufficient smoothness of a coating containing such particles. For drug delivery systems (DDS), the presence of large particles in larger proportions may lead to variations in the amount of drug incorporated into each particle and may also lead to variations in controlled release time and temperature.

For hollow silica nanoparticles prepared from the core-shell silica nanoparticles, for example, by calcination, the presence of large particles in larger proportions is undesirable because they tend to cause variations in light scattering and decrease the transparency.

The present invention will now be described in greater detail.

Copolymer (A) Containing Aliphatic Polyamine Chain (a1) Containing Primary Amino Groups and/or Secondary Amino Groups and Hydrophobic Organic Segment (a2)

In the present invention, the aliphatic polyamine chain (a1) containing primary amino groups and/or secondary amino groups in the copolymer (A) may be any polyamine chain that can be dissolved in an aqueous medium to form aggregates including a core composed of the hydrophobic organic segment (a2). Examples of such polyamine chains include branched polyethyleneimine chains, linear polyethyleneimine chains, and polyallylamine chains. Branched polyethyleneimine chains are desirable for efficient manufacture of the target silica nanoparticles. The polyamine chain (a1) portion may have any molecular weight that allows aggregates to form in balance with the hydrophobic organic segment (a2). Preferably, the polyamine chain portion contains 5 to 10,000 repeating polymer units, particular preferably 10 to 8,000 repeating polymer units, since such a copolymer is suitable for forming aggregates.

The aliphatic polyamine chain (a1) portion may have any molecular structure. Examples of suitable molecular structures include linear, branched, dendritic, star-shaped, and comb-shaped structures. Branched polyethyleneimine chains are preferred for efficient formation of aggregates serving as a template for silica precipitation and for other reasons such as manufacturing costs.

The backbone of the aliphatic polyamine chain (a1) containing primary amino groups and/or secondary amino groups may be a polyamine chain composed of amine units of a single type or a polyamine chain backbone of amine units of two or more types (copolymer). The backbone of the aliphatic polyamine chain (a1) may also contain polymer units other than amine units in a proportion that allows aggregates to form in an aqueous medium. Preferably, other polymer units are present in the amine backbone of the aliphatic polyamine chain (a1) in a proportion of 50 mol % or less, more preferably 30 mol % or less, most preferably 15 mol % or less, since such a copolymer is suitable for forming aggregates.

The hydrophobic organic segment (a2) in the copolymer (A) may be any hydrophobic organic segment that can form stable aggregates including a core composed of the hydrophobic organic segment (a2) in an aqueous medium by hydrophobic interaction. Examples of such hydrophobic organic segments include segments composed of alkyl compounds such as alkyl glycidyl ethers and segments composed of hydrophobic polymers such as polyacrylates, polystyrenes, and polyurethanes. The alkyl compounds preferably have an alkylene chain of 5 or more carbon atoms, more preferably 10 or more carbon atoms. The hydrophobic polymer chains may have any length that allows nano-sized aggregates to be stabilized. Preferably, the polymer chain contains 5 to 10,000 repeating polymer units, particular preferably 5 to 1,000 repeating polymer units, since such a copolymer is suitable for forming aggregates.

The hydrophobic organic segment (a2) may be joined to the aliphatic polyamine (a1) by any stable chemical bond. For example, the hydrophobic organic segment (a2) may be coupled to an end of the polyamine or may be grafted to the polyamine backbone.

The polyamine chain (a1) may have joined thereto either a single hydrophobic organic segment (a2) or a plurality of hydrophobic organic segments (a2).

The aliphatic polyamine chain (a1) and the hydrophobic organic segment (a2) may be present in the copolymer (A) in any proportion that allows stable aggregates to form in an aqueous medium. To facilitate formation of aggregates, the polyamine chain is preferably present in a proportion of 10% to 90% by mass, more preferably 30% to 70% by mass, most preferably 40% to 60% by mass.

The copolymer (A) used in the present invention can be modified with various functional molecules. The copolymer (A) may be modified either on the aliphatic polyamine chain (a1) or on the hydrophobic organic segment (a2). The copolymer (A) may be modified with any functional molecule that allows stable aggregates to form in an aqueous medium. Aggregates of such a modified copolymer (A) can be used as a template for silica precipitation to form core-shell silica nanoparticles having any functional molecule introduced therein. In view of this, it is particularly preferred to modify the copolymer (A) with fluorescent compounds. The use of fluorescent compounds provides fluorescent core-shell silica nanoparticles suitable for applications in various fields.

### Core-Shell Silica Nanoparticles

The core-shell silica nanoparticles according to the present invention include a core layer based on the hydrophobic organic segment (a2) portion and a shell layer composed of a hybrid based on the aliphatic polyamine chain (a1) and silica (B). By “based on”, it is meant that no components other than the copolymer (A) and the silica (B) are present unless any third component is deliberately introduced and that, for example, when aggregates of the copolymer (A) are formed in an aqueous medium, the polyamine chain (a1) may be partially present in the core portion, or the hydrophobic organic segment (a2) may be partially present in the shell layer portion. In particular, the shell layer of the particles is an organic-inorganic hybrid containing a silica matrix hybridized with the aliphatic polyamine chain (a1).

The core-shell silica nanoparticles provided by the present invention preferably have an average particle size of 5 to 100 nm, more preferably 5 to 30 nm, even more preferably 5 to 20 nm, particularly preferably 5 to less than 20 nm, most preferably 5 to 15 nm. The particle size of the core-shell silica nanoparticles can be controlled depending on, for example, the manner of preparation of aggregates (e.g., the type, composition, and molecular weight of the copolymer (A) used), the type of silica source, and the sol-gel reaction conditions. The core-shell silica nanoparticles, which are formed via molecular self-assembly, have significantly good monodispersity. Specifically, the width of the particle size distribution can be controlled to within ±15% from the average particle size.

The core-shell silica nanoparticles according to the present invention may be spherical or string-shaped with an aspect ratio of 2 or more. Core-shell silica nanoparticles each including a plurality of cores can also be synthesized. The properties such as the shape and structure of the particles can be controlled depending on, for example, the composition of the copolymer (A), the manner of preparation of aggregates, the type of silica source, and the sol-gel reaction conditions.

Silica may be present in the core-shell silica nanoparticles according to the present invention in varying amounts within a certain range depending on, for example, the reaction conditions. Typically, silica may be present in an amount of 30% to 95% by mass, preferably 60% to 90% by mass, of the total amount of core-shell silica nanoparticles. The silica content can be changed depending on, for example, the amount of aliphatic polyamine chain (a1) present in the copolymer (A) used in the sol-gel reaction, the amount of aggregates, the type and amount of silica source, and the sol-gel reaction time and temperature.

Organosilanes such as polysilsesquioxanes can be incorporated into the core-shell silica nanoparticles according to the present invention by performing a sol-gel reaction of an organosilane after silica precipitation. Such core-shell silica nanoparticles containing organosilanes such as polysilsesquioxanes have good monodispersity and exhibit high sol stability in a solvent. Once dried, the core-shell silica nanoparticles can be redispersed in a medium. This contrasts with the nature of existing fine silica particles, which are difficult to redisperse into particles once a dispersion containing the fine silica particles is dried. Fine silica particles prepared by existing processes such as the Stober process are difficult to redisperse in a medium unless the surface of the resulting fine particles is chemically modified with materials such as surfactants, and processes such as pulverization are often required to form nano-level ultrafine particles because drying results in, for example, secondary aggregation.

The core-shell silica nanoparticles according to the present invention allow metal ions to be highly concentrated and adsorbed onto the aliphatic polyamine chain (a1) present in the silica matrix of the shell layer. The core-shell silica nanoparticles according to the present invention also allow various ionic materials, such as anionic biological materials, to be adsorbed and immobilized on the aliphatic polyamine chain (a1), which is cationic. The hydrophobic organic segment (a2) portion in the copolymer (A) can impart various functions to the core-shell silica nanoparticles according to the present invention since various hydrophobic organic segments can be selected depending on the functionality and their structures can also be easily controlled.

Such functions can be imparted, for example, by immobilizing fluorescent materials. For example, if a small amount of fluorescent material, such as a pyrene or porphyrin, is introduced into the aliphatic polyamine chain (a1), its functional residue is incorporated into the shell layer of the silica nanoparticles. Fluorescent dyes such as porphyrins, phthalocyanines, and pyrenes can also be incorporated into the shell layer of the silica nanoparticles by adding a small amount of a fluorescent material containing an acidic group, such as a carboxylic acid or sulfonic acid group, to the basic groups of the aliphatic polyamine chain (a1). Similarly, functional materials can be selectively incorporated into the core layer of the silica nanoparticles by selectively immobilizing functional materials on the hydrophobic organic segment (a2) before aggregate formation and silica precipitation.

The silica nanoparticles according to the present invention can be dried and used in the form of a powder and can be used as a filler for other compounds such as resins. After drying, the powder can be redispersed in a solvent to form a dispersion or sol for addition to other compounds.

### Method for Manufacturing Core-Shell Silica Nanoparticles

A method for manufacturing the core-shell silica nanoparticles according to the present invention includes a step of precipitating the silica (B) in the presence of aggregates, formed in an aqueous medium, of the copolymer (A) containing the aliphatic polyamine chain (a1) containing primary amino groups and/or secondary amino groups and the hydrophobic organic segment (a2). This method may further include a step of performing a sol-gel reaction of an organosilane after the step of precipitating silica to introduce a polysilsesquioxane.

The method of manufacture according to the present invention begins by dissolving the copolymer (A) containing the aliphatic polyamine chain (a1) containing primary amino groups and/or secondary amino groups and the hydrophobic organic segment (a2) in an aqueous medium. The copolymer (A) can form aggregates having a core-shell structure via self-assembly. These aggregates include a core based on the hydrophobic organic segment (a2) and a shell layer based on the aliphatic polyamine chain (a1). The hydrophobic organic segment (a2) will form stable aggregates in the medium because of its hydrophobic interaction.

The aqueous medium used to form the aggregates may be any medium containing water in which stable aggregates can be formed. Examples of such aqueous media include water and mixtures of water with water-soluble solvents. A mixture of water with a water-soluble solvent may be used in a volume ratio of water to the water-soluble solvent of 0.5:9.5 to 3:7, preferably 0.1:9.9 to 5:5. Although mixtures of water with alcohols may be used for reasons such as productivity, environment, and cost, water is preferably used alone.

The copolymer (A) may be present in the aqueous medium in any concentration that does not essentially result in coalescence of aggregates to each other, typically 0.05% to 15% by mass, preferably 0.1% to 10% by mass, most preferably 0.2% to 5% by mass.

Whereas the formation of aggregates of the copolymer (A) via self-assembly in an aqueous medium in the present invention is a simple process, the polyamine chain (a1) in the shell layer of the aggregates may be crosslinked using organic compounds having two or more functional groups. This gives a product similar to aggregates. Examples of such organic compounds include aldehyde compounds, epoxy compounds, unsaturated double-bond containing compounds, and carboxyl-containing compounds having two or more functional groups.

The method for manufacturing the core-shell silica nanoparticles according to the present invention includes, after the step of forming aggregates, the step of forming silica, specifically, the step of effecting a sol-gel reaction of a silica source using the aggregates as a template in the presence of water. This method may further include, after silica precipitation, the step of effecting a sol-gel reaction of an organosilane to incorporate a polysilsesquioxane into the core-shell silica nanoparticles.

The sol-gel reaction can be performed by mixing an aggregate dispersion with a silica source to easily form core-shell silica nanoparticles. Examples of silica sources include water glass, tetraalkoxysilanes, and tetraalkoxysilane oligomers.

Examples of tetraalkoxysilanes include tetramethoxysilane, tetraethoxysilane, tetrapropoxysilane, tetrabutoxysilane, and tetra-t-butoxysilane.

Other examples include tetramethoxysilane tetramer, tetramethoxysilane heptamer, tetraethoxysilane pentamer, and tetraethoxysilane decamer.

The sol-gel reaction that gives core-shell silica nanoparticles does not occur in the continuous phase of the solvent; it proceeds selectively only in the aggregate domains. The sol-gel reaction may therefore be effected under any conditions that do not result in dissociation of the aggregates.

The silica source may be used in the sol-gel reaction in any amount relative to the aggregates. The proportion of the silica source to the aggregates can be selected depending on the composition of the target core-shell silica nanoparticles. If a polysilsesquioxane structure is introduced into the core-shell silica nanoparticles using an organosilane after silica precipitation, the organosilane is preferably used in an amount of 50% by mass or less, more preferably 30% by mass or less, of the silica source.

Examples of organosilanes that can be used to introduce a polysilsesquioxane into the nanoparticles include alkyltrialkoxysilanes, dialkylalkoxysilanes, and trialkylalkoxysilanes.

Examples of alkyltrialkoxysilanes include methyltrimethoxysilane, methyltriethoxysilane, ethyltrimethoxysilane, ethyltriethoxysilane, n-propyltrimethoxysilane, n-propyltriethoxysilane, isopropyltrimethoxysilane, isopropyltriethoxysilane, 3-chloropropyltrimethoxysilane, 3-chloropropyltriethoxysilane, vinyltrimethoxysilane, vinyltriethoxysilane, 3-glycidoxypropyltrimethoxysilane, 3-glycidoxypropyltriethoxysilane, 3-aminopropyltrimethoxysilane, 3-aminopropyltriethoxysilane, 3-mercaptopropyltrimethoxysilane, 3-mercaptotriethoxysilane, 3,3,3-trifluoropropyltrimethoxysilane, 3,3,3-trifluoropropyltriethoxysilane, 3-methacryloxypropyltrimethoxysilane, 3-methacryloxypropyltriethoxysilane, phenyltrimethoxysilane, phenyltriethoxysilane, p-chloromethylphenyltrimethoxysilane, and p-chloromethylphenyltriethoxysilane.

Examples of dialkylalkoxysilanes include dimethyldimethoxysilane, dimethyldiethoxysilane, diethyldimethoxysilane, and diethyldiethoxysilane.

Examples of trialkylalkoxysilanes include trimethylmethoxysilane and trimethylethoxysilane.

The sol-gel reaction may be performed at any temperature. For example, the reaction temperature is preferably 0° C. to 90° C., more preferably 10° C. to 40° C. To efficiently manufacture the core-shell silica nanoparticles, the reaction temperature is preferably set to 15° C. to 30° C.

The sol-gel reaction may be performed for any period of time, varying from 1 minute to several weeks. For water glass and methoxysilanes, which are alkoxysilanes with high reactivity, the reaction time may be 1 minute to 24 hours, preferably 30 minutes to 5 hours, which results in a higher reaction efficiency. For ethoxysilanes and butoxysilanes, which have low reactivity, the sol-gel reaction time is preferably 5 hours or more, and even about 1 week is preferred. The sol-gel reaction of the organosilane is preferably performed for 3 hours to 1 week, depending on the reaction temperature.

The method of manufacture described above provides core-shell silica nanoparticles with uniform particle size that do not aggregate with each other. Depending on the manufacturing conditions and the target particle size, the width of the particle size distribution of the resulting core-shell silica nanoparticles can be controlled to within ±15%, or within ±10% under preferred conditions, from the target particle size (average particle size).

As described above, unlike existing core-shell silica nanoparticles, the method for manufacturing the core-shell silica nanoparticles according to the present invention provides core-shell silica nanoparticles with good monodispersity that have an extremely small particle size by introducing the aliphatic polyamine chain (a1) containing primary amino groups and/or secondary amino groups, which are highly reactive, into the silica matrix of the shell layer. The resulting core-shell silica nanoparticles can be modified with polysilsesquioxanes for use in applications such as resin fillers and abrasive fillers.

The core-shell silica nanoparticles according to the present invention allow the immobilization and concentration of various materials on the aliphatic polyamine chain (a1) containing primary amino groups and/or secondary amino groups, which are highly reactive, present in the shell layer in the form of a hybrid with the silica matrix. The core-shell silica nanoparticles according to the present invention also allow the functionalization of the hydrophobic organic segment (a2) present in the core layer. Allowing the selective immobilization and concentration of metals and biological materials in the nano-sized spheres and the modification of the interior of the particles with functional molecules, the core-shell silica nanoparticles according to the present invention are useful in various fields, including the fields of electronic materials, biology, and environmentally compatible products.

The method for manufacturing the core-shell silica nanoparticles according to the present invention is much easier than known and widely used methods such as the StÖber process and provides core-shell silica nanoparticles that cannot be produced by the Stober process. This method is expected to find a wide range of applications irrespective of the industry and field. The core-shell silica nanoparticles according to the present invention are useful not only in the general applications of silica materials, but also in the applications of polyamines.

Hollow silica nanoparticles manufactured from the core-shell silica nanoparticles described above and a method for manufacturing such hollow silica nanoparticles will now be described in detail.

### Method for Manufacturing Hollow Silica Nanoparticles

The method for manufacturing the hollow silica nanoparticles according to the present invention includes the following three steps: steps (1) and (2), in which core-shell silica nanoparticles are manufactured, and step (3), in which the core is removed after steps (1) and (2).

(1) a step of mixing the copolymer (A) containing the aliphatic polyamine chain (a1) containing primary amino groups and/or secondary amino groups and the hydrophobic organic segment (a2) with an aqueous medium to form aggregates including a core layer based on the hydrophobic organic segment (a2) and a shell layer based on the aliphatic polyamine chain (a1);

(2) a step of adding a silica source (b) to the aqueous medium containing the aggregates formed in step (1) and effecting a sol-gel reaction of the silica source using the aggregates as a template to precipitate silica (B), thereby forming core-shell silica nanoparticles; and

(3) a step of removing the copolymer (A) from the core-shell silica nanoparticles formed in step (2).

After core-shell silica nanoparticles are formed as a precursor in step (2), the copolymer (A) is removed from the nanoparticles in step (3) to obtain the target hollow silica nanoparticles.

The copolymer (A) can be removed by processes such as calcination and washing with solvents. calcination in a firing furnace is preferred since the copolymer (A) can be completely removed.

Although the calcination process may be performed either by high-temperature calcination in air and oxygen or by high-temperature calcination in an inert gas such as nitrogen or helium, calcination in air is generally preferred.

The calcination temperature is preferably 300° C. or higher, particularly preferably 300° C. to 1,000° C., since the copolymer (A) starts decomposing thermally around 300° C.

Core-shell silica nanoparticles containing a polysilsesquioxane may be calcined at any temperature below which the polysilsesquioxane decomposes thermally. For example, if core-shell silica nanoparticles containing polymethylsilsesquioxane are calcined at 400° C., the copolymer (A) can be removed while the polymethylsilsesquioxane remains in the resulting hollow silica nanoparticles.

The method of manufacture according to the present invention provides ultrafine hollow silica nanoparticles with good monodispersity. The resulting hollow silica nanoparticles have an outer diameter of 5 to 30 nm and an inner diameter of 1 to 30 nm. In particular, as described above, preferred ultrafine hollow silica nanoparticles provided by the method of manufacture according to the present invention have an outer diameter of 5 to 20 nm and an inner diameter of 1 to 10 nm. Such ultrafine hollow silica nanoparticles cannot be manufactured by existing methods for manufacturing nano-sized hollow silica particles, for example, those using polymer latex nanoparticles or block polymer micelles as a template. Hollow silica nanoparticles containing a polysilsesquioxane can also be manufactured.

The hollow silica nanoparticles provided by the present invention may each include one or more cores (hollow structures). The hollow silica nanoparticles may be spherical or string-shaped with an aspect ratio of 2 or more. The properties such as the particle size, structure, and shape of the hollow silica nanoparticles can be controlled depending on, for example, the manufacturing conditions of the precursor core-shell silica nanoparticles.

The hollow silica nanoparticles provided by the present invention can be used in the form of a powder and can be used as a filler for other compounds such as resins. After drying, the powder can be redispersed in a solvent to form a dispersion or sol for addition to other compounds.

The method for manufacturing the hollow silica nanoparticles according to the present invention, which involves the use of a template designed based on molecular self-assembly and a sol-gel reaction mimicking biogenic silica, is much simpler and easier than known and widely used methods of manufacture and provides ultrafine hollow silica nanoparticles that cannot be produced by existing methods for manufacturing hollow silica using nanoparticles as a template. This method is expected to find a wide range of applications irrespective of the industry and field. In particular, the hollow silica nanoparticles according to the present invention are useful in the fields of antireflection materials, low-dielectric-constant materials, heat insulation materials, and drug delivery systems.

The method for manufacturing the hollow silica nanoparticles according to the present invention is environmentally compatible since the step of forming the aggregates of the copolymer (A) and the step of performing the sol-gel reaction of the silica source (b) can be performed in water within a short period of time. The method for manufacturing the hollow silica nanoparticles is also useful since the preparation of the aggregates of the copolymer (A) and the removal of the copolymer (A) from the core-shell silica nanoparticles can be easily performed using general-purpose equipment.

### EXAMPLES

The present invention is further illustrated by the following examples, although these examples are not intended to limit the present invention. Unless otherwise specified, percentages are by mass.

Assessment of Chemical Bonds between Copolymer and Silica by NMR

A synthesized copolymer (A) was examined by ¹H-NMR (AL300 available from JEOL Ltd., 300 Hz) to determine its chemical structure. A core-shell silica nanoparticle powder was also examined by solid-state ²⁹Si CP/MAS-NMR (JNM-ECA600 available from JEOL Ltd., 600 Hz) to assess the degree of condensation of silica (Q4, Q3, and Q2).

Examination under Transmission Electron Microscope (TEM)

A dispersion of synthesized silica nanoparticles was diluted with ethanol and was placed on a carbon-deposited copper grid. The sample was examined under JEM-2200FS available from JEOL Ltd.

### Assessment of Particle Size and Core-Shell Structure by X-Ray Small-Angle Scattering

A silica nanoparticle powder was examined by small-angle scattering (TTRII available from Rigaku Corporation), and the resulting scattering curve was analyzed by NANO-Solver to estimate the particle size.

By “good monodispersity”, specifically, it is meant that the width of the particle size distribution expressed by equation (1) below is within 15%.

Width of particle size distribution=(standard deviation of particle size)×100/average particle size (average of particle sizes) (1)

The average particle size and standard deviation of the particles were calculated from the diameters of 100 particles manufactured under the same conditions as measured under an electron microscope.

This assessment procedure was used for both core-shell silica nanoparticles and hollow silica nanoparticles, described below.

### Assessment of Composition by TGA

A silica nanoparticle powder was examined by TGA (TG/DTA6300 available from SII NanoTechnology Inc.). The composition of the particles was estimated from the decrease in mass in the range of 150° C. to 800° C.

### Calcination Process

Calcination was performed in an ARF-100K ceramic electric tubular furnace equipped with an AMF-2P temperature controller available from Asahi Rika Co., Ltd.

### Measurement of Specific Surface Area

The specific surface area was measured by nitrogen gas adsorption/desorption using a Trisstar 3000 analyzer available from Micromeritics Instrument Corporation. The pore size distribution was estimated from a plot of pore volume fraction versus pore size.

### Example Synthesis 1

### Synthesis of Copolymer (A-1)

Into 40 mL of ethanol were dissolved 1.5 g of branched polyethyleneimine (SP003 available from Nippon Shokubai Co., Ltd., average molecular weight: 300) and 0.5 g of glycidyl hexadecyl ether (reagent available from Aldrich, hereinafter “EP-C16”). The solution was reacted at 75° C. for 24 hours. The ethanol was removed, and the reaction product was dried in a vacuum at 60° C. to obtain a copolymer (hereinafter “A-1”). The ¹H-NMR spectrum of the reaction product showed a broad signal derived from the protons adjacent to the ether oxygen (3.0 to 4.0 ppm), demonstrating that the copolymer (A-1) was formed.

Other copolymers (hereinafter “A-2” to “A-13”) were synthesized by the procedure described above. The mass proportions of the raw materials used are shown in Table 1, in which SP003, SP006, SP012, SP018, SP200, and P1000 are branched polyethyleneimines (available from Nippon Shokubai Co., Ltd.) having average molecular weights of 300, 600, 1,200, 1,800, 10,000, and 70,000, respectively. The polyallylamine (PAA) has an average molecular weight of 15,000 (available from Nitto Boseki Co., Ltd.). The 2-ethylhexyl glycidyl ether is a reagent available from Tokyo Chemical Industry Co., Ltd. (hereinafter “EP-C8”).

TABLE 1

| Copolymer | SP003 | SP006 | SP012 | SP018 | SP200 | P1000 | PAA | EP-C₁₆ | EP-C₈ |
|---|---|---|---|---|---|---|---|---|---|
| A-1 | 1.0 | | | | | | | 1.0 | |
| A-2 | | 1.5 | | | | | | 0.5 | |
| A-3 | | 1.0 | | | | | | 1.0 | |
| A-4 | | | 1.5 | | | | | 0.5 | |
| A-5 | | | 1.0 | | | | | 1.0 | |
| A-6 | | | | 1.5 | | | | 0.5 | |
| A-7 | | | | 1.0 | | | | 1.0 | |
| A-8 | | | | | 1.5 | | | 0.5 | |
| A-9 | | | | | 1.0 | | | 1.0 | |
| A-10 | | | | | 1.5 | | | | 0.5 |
| A-11 | | | | | | 1.5 | | 0.5 | |
| A-12 | | | | | | 1.0 | | 1.0 | |
| A-13 | | | | | | | 1.5 | 0.5 | |

### Example 1

### Synthesis of Core-Shell Silica Nanoparticles

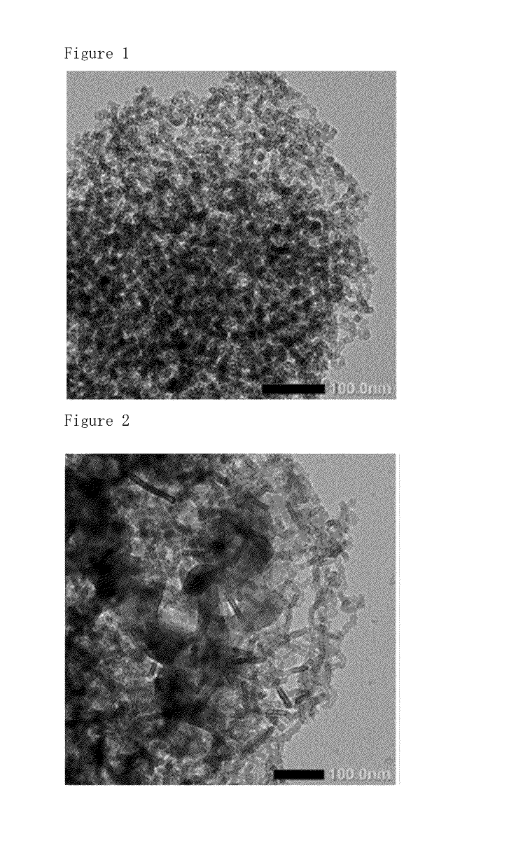

A mixture of 0.05 g of the copolymer (A-8) and 5 mL of water was stirred at 80° C. for 24 hours to form aggregates. To the aggregate dispersion, 0.50 mL of MS51 (methoxysilane tetramer) was added as a silica source. The resulting dispersion was stirred at room temperature for 4 hours. The reaction product was washed with ethanol and was dried to obtain a powder. Estimation from TGA measurement data showed that the powder had an organic content of 17.3%. TEM examination confirmed that the resulting powder had a core-shell structure (FIG. 1). The 3.5 nm diameter core in the center, which looks light, is assumed to be composed of a hydrophobic organic segment with relatively low electron density. The 4 nm thick shell layer, which looks dark, is assumed to be composed of a hybrid of an aliphatic polyamine and silica with high electron density. The resulting powder was composed of spherical particles with good monodispersity that had a particle size of not more than 11 nm.

The powder obtained in Example 1 was examined by X-ray small-angle scattering. From the results of scattering, the particle size, core size, and shell thickness of the sample were calculated to be 11.9 nm, 3.1 nm, and 4.3 nm, respectively. These results substantially match the results of TEM examination.

The powder was also examined by ²⁹Si CP/MAS-NMR to assess the chemical bonds of silica in the powder. As a result, the integral areas of Q4, Q3, and Q2 in the silica network were determined to be 45.5%, 51.9%, and 2.6%, respectively. The predominant presence of Q4 and Q3 suggests that the polyamine forming the shell of the aggregates of the copolymer (C) functions as a catalyst and scaffold for a sol-gel reaction. The above results demonstrate that the powder obtained in Example 1 was composed of core-shell silica nanoparticles according to the present invention.

### Examples 2 to 16

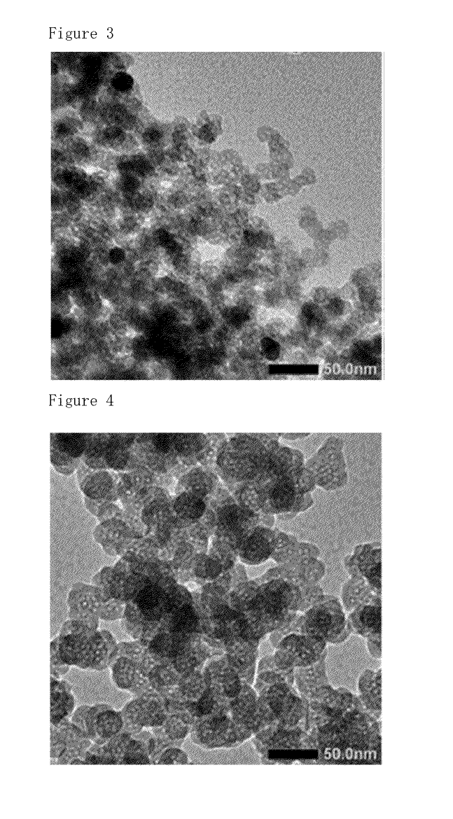

Core-shell silica nanoparticles were synthesized using the method for preparing aggregates and the conditions for the sol-gel reaction of the silica source in Example 1. The results are shown in Table 2. The sol-gel reaction was effected at room temperature for 4 hours. The average size and the shape were determined by TEM examination. FIGS. 2, 3, 4, and 5 show TEM images of the core-shell silica nanoparticles of Examples 5, 8, 11, and 12, respectively.

TABLE 2

| | | Aggre- | | | Copol- | |
|---|---|---|---|---|---|---|
| | | gate | | | ymer | |
| Exam- | Copol- | disper- | MS51 | Yield | content | Size (nm)/ |
| ple | ymer | sion | (mL) | (mg) | (%) | shape |
| 1 | A-8 | 1% | 0.5 | 319 | 17.3 | 11 |
| | | 5 mL | | | | Core-shell |
| 2 | A-8 | 1% | 0.25 | 175 | 19.0 | 10 |
| | | 5 mL | | | | Core-shell |
| 3 | A-8 | 0.2% | 0.25 | 89 | 13.1 | 10 |
| | | 5 mL | | | | Core-shell |
| 4 | A-8 | 5% | 2.0 | 1,197 | 18.2 | 12 |
| | | 2 mL | | | | Core-shell |
| 5 | A-9 | 1% | 0.5 | 178 | 28.4 | String-shaped, |
| | | 5 mL | | | | 15 in diameter |
| | | | | | | Core-shell |
| 6 | A-7 | 1% | 0.5 | 277 | 20.4 | 10 |
| | | 5 mL | | | | Core-shell |
| 7 | A-6 | 1% | 0.5 | 301 | 19.2 | 10 |
| | | 5 mL | | | | Core-shell |
| 8 | A-5 | 1% | 0.5 | 284 | 18.9 | 20 |
| | | 5 mL | | | | Multicore |
| 9 | A-4 | 1% | 0.5 | 281 | 20.3 | 10 |
| | | 5 mL | | | | Core-shell |
| 10 | A-3 | 1% | 0.5 | 269 | 19.7 | 35 |
| | | 5 mL | | | | Multicore-shell |
| 11 | A-2 | 1% | 0.5 | 256 | 21.8 | 50 |
| | | 5 mL | | | | Multicore-shell |
| 12 | A-1 | 1% | 0.5 | 260 | 20.0 | 60 |
| | | 5 mL | | | | Multicore-shell |
| 13 | A-11 | 1% | 0.5 | 300 | 18.3 | 12 |
| | | 5 mL | | | | Core-shell |
| 14 | A-12 | 1% | 0.5 | 230 | 24.1 | 13 |
| | | 5 mL | | | | Core-shell |
| 15 | A-13 | 1% | 0.5 | 290 | 18.5 | 16 |
| | | 5 mL | | | | Core-shell |
| 16 | A-10 | 0.5% | 0.3 | 95 | 12.0 | 9 |
| | | 5 mL | | | | Core-shell |

In Table 2, “diameter” should be read as “major axis”.

### Comparative Example 1

Aggregate formation and silica precipitation were performed as in Example 1 except that branched polyethyleneimine (SP200, available from Nippon Shokubai Co., Ltd., average molecular weight: 10,000) was used alone without a hydrophobic organic segment. The entire dispersion gelled. Core-shell silica nanoparticles cannot be formed because the branched polyethyleneimine, which has no hydrophobic segment joined thereto, cannot form aggregates serving as a template for the sol-gel reaction of the silica source.

### Comparative Example 2

Hydrophilic polyethylene glycol (average molecular weight: 5,000) was joined to branched polyethyleneimine (average molecular weight: 10,000) in accordance with the method disclosed in Japanese Unexamined Patent Application Publication No. 2010-118168 (Example Synthesis 1) (in a molar ratio of ethyleneimine units to ethylene glycol units of 1:3). The resulting copolymer was used to perform aggregate formation and silica precipitation as in Example 1. The entire dispersion gelled. Core-shell silica nanoparticles cannot be formed because the branched polyethyleneimine, which has hydrophilic polyethylene glycol joined thereto, cannot form core-shell aggregates including a hydrophobic core by hydrophobic interaction in water, resulting in the gelation of the entire dispersion.

### Example 17

### Synthesis of Core-Shell Silica Nanoparticles Under Neutral Conditions

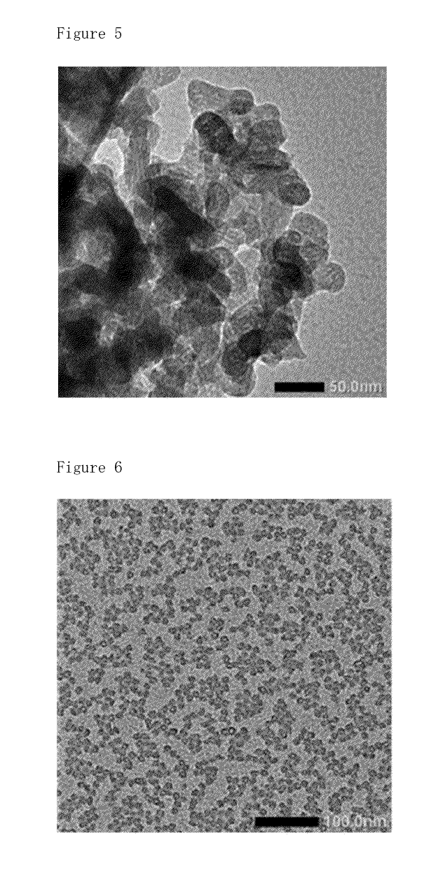

A mixture of 0.05 g of the copolymer (A-1) and 5 mL of water was stirred at 80° C. for 24 hours to form aggregates. The aggregate dispersion of the copolymer (A-1) was adjusted to around pH 7.0 with aqueous hydrochloric acid. To the resulting aggregate dispersion, 0.50 mL of MS51 was added as a silica source. The mixture was stirred at room temperature for 4 hours to obtain a nanoparticle sol. The sol was transparent and had high sol stability at room temperature. The sol was diluted with ethanol to prepare a sample for TEM examination. TEM examination confirmed that core-shell silica nanoparticles with good dispersibility were formed (FIG. 6). The particle size, the core size, and the shell layer thickness were 10 nm, 3 nm, and 4 nm, respectively.

### Example 18

### Synthesis of Polysilsesquioxane-Modified Core-Shell Silica Nanoparticles

After the silica precipitation in Example 1, 0.1 mL of trimethylmethoxysilane was added to the dispersion. The resulting dispersion was stirred at room temperature for 24 hours. The reaction product was washed with ethanol and was dried to obtain polysilsesquioxane-modified core-shell silica nanoparticles. TEM examination confirmed that spherical core-shell silica nanoparticles with good monodispersity that had a particle size of 13 nm were formed.

### Synthesis of Hollow Silica Nanoparticles

### Example 19

### Synthesis of Hollow Silica Nanoparticles from Core-Shell Silica Nanoparticles

A mixture of 0.05 g of the copolymer synthesized in Example Synthesis 1 (A-8: a copolymer of 1.5 g of SP200 branched polyethyleneimine (available from Nippon Shokubai Co., Ltd., average molecular weight: 10,000) and 0.5 g of glycidyl hexadecyl ether) and 5 mL of water was stirred at 80° C. overnight to form aggregates. To the aggregate dispersion, 0.50 mL of MS51 (methoxysilane tetramer) was added as a silica source. The resulting dispersion was stirred at room temperature for 4 hours. The reaction product was washed with ethanol and was dried to obtain core-shell silica nanoparticles. The yield was 0.32 g.

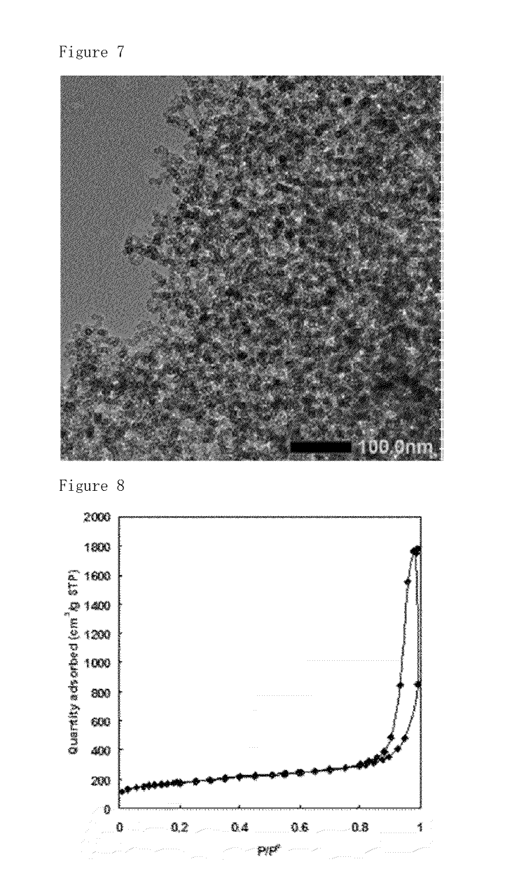

In an alumina crucible were placed 0.1 g of the thus-obtained core-shell silica nanoparticles, and they were calcined in an electric furnace. The furnace was heated to 600° C. over 5 hours, was maintained at that temperature for 3 hours, and was allowed to cool to remove the copolymer (A-1). The yield was 0.083 g. TEM examination confirmed that the resulting silica nanoparticles had a hollow structure (FIG. 7). The pore in the center had a diameter of 3.5 nm, and the shell layer had a thickness of 4 nm. The resulting hollow silica nanoparticles were spherical particles with good monodispersity that had an average particle size of not more than 11 nm.

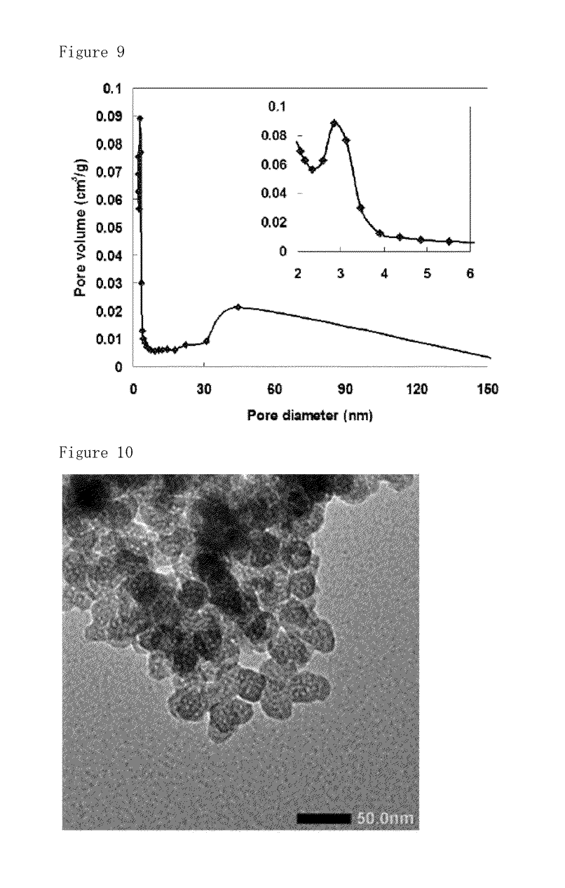

The resulting powder had a specific surface area of 593.5 m²/g. The isothermal curves and pore size distribution of the powder are shown in FIGS. 8 and 9, respectively. FIG. 9 shows that the peak pore size was 3.0, which reflects the pore size of the silica particles and substantially matches the inner diameter (3.5 nm) determined by TEM examination.

The hollow silica nanoparticles were also examined by ²⁹Si CP/MAS-NMR to assess the chemical bonds of silica in the hollow silica nanoparticles. As a result, the integral areas of Q4, Q3, and Q2 in the silica network were 21.9%, 65.9%, and 12.2%, respectively.

### Example 20

### Synthesis of Polysilsesquioxane-Containing Core-Shell Silica Nanoparticles

A mixture of 0.10 g of the copolymer (A-8) synthesized in Example Synthesis 1 and 10 mL of water was stirred at 80° C. for 24 hours to form aggregates. To the aggregate dispersion, 0.8 mL of MS51 (methoxysilane tetramer) was added as a silica source. The resulting mixture was stirred at room temperature for 4 hours, and 0.2 mL of trimethylmethoxysilane was then added. The resulting dispersion was stirred at room temperature for 24 hours. The reaction product was washed with ethanol and was dried to obtain polysilsesquioxane-containing core-shell silica nanoparticles.

### Synthesis of Polysilsesquioxane-Containing Hollow Silica Nanoparticles

The thus-obtained polysilsesquioxane-containing core-shell silica nanoparticles were placed in an alumina crucible and were calcined in an electric furnace. The furnace was heated to 400° C. over 2 hours, was maintained at that temperature for 1 hour, and was allowed to cool to obtain polysilsesquioxane-containing hollow silica nanoparticles containing no copolymer (A-1). TEM examination confirmed that the resulting nanoparticles had a particle size of not more than 11 nm and had a hollow structure with an inner diameter of 3.5 nm.

### Example 21

A mixture of 0.05 g of the copolymer synthesized in Example 1 (A-2: a copolymer of 1.5 g of SP006 branched polyethyleneimine (available from Nippon Shokubai Co., Ltd., average molecular weight: 600) and 0.5 g of glycidyl hexadecyl ether) and 5 mL of water was stirred at 80° C. for 56 hours to form aggregates. To the aggregate dispersion, 0.50 mL of MS51 (methoxysilane tetramer) was added as a silica source. The resulting dispersion was stirred at room temperature for 4 hours. The reaction product was washed with ethanol and was dried to obtain core-shell silica nanoparticles. The yield was 0.26 g.

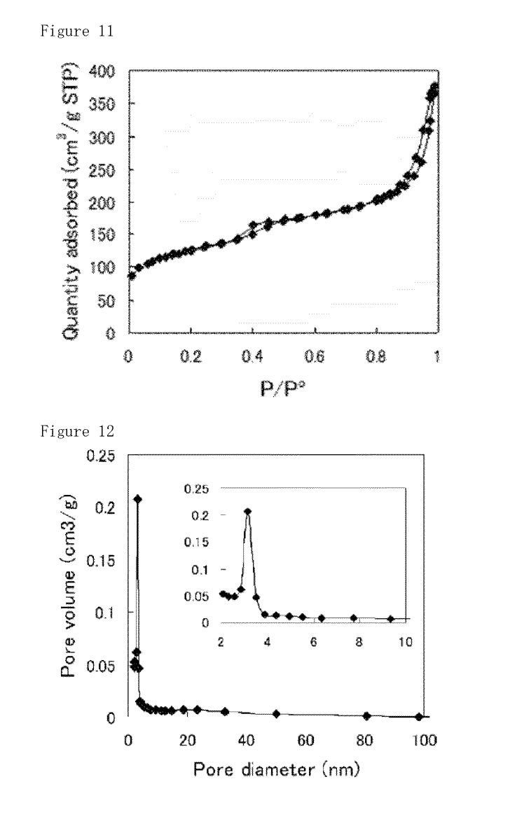

As in Example 1, 0.1 g of the thus-obtained core-shell silica nanoparticles were calcined. The yield was 0.081 g. TEM examination confirmed that the resulting silica nanoparticles had a hollow structure (FIG. 10). The TEM examination also confirmed that the particles had an outer diameter of not more than 50 nm and included a plurality of 3.5 nm diameter pores in the center thereof. The resulting hollow silica nanoparticle powder had a specific surface area of 419.4 m2/g. The isothermal curves and pore size distribution of the powder are shown in FIGS. 11 and 12, respectively. FIG. 12 shows that the peak pore size was 3.2, which reflects the pore size of the silica particles and substantially matches the pore size (3.5 nm) determined by TEM examination.

For all silica nanoparticles of Examples 1 to 4 and 6 to 21, including both the core-shell silica nanoparticles and the hollow silica nanoparticles, the width of the particle size distribution was within 10%. Such good monodispersity will provide the technical advantages described above.

### Example 22

### Synthesis of String-Shaped Core-Shell Silica Nanoparticles

A mixture of 0.05 g of the copolymer synthesized in Example Synthesis 1 (A-9: a copolymer of 1.0 g of SP200 branched polyethyleneimine (available from Nippon Shokubai Co., Ltd., average molecular weight: 10,000) and 1.0 g of glycidyl hexadecyl ether) and 5 mL of water was stirred at 80° C. for 24 hours to form aggregates. To the aggregate dispersion, 0.50 mL of MS51 (methoxysilane tetramer) was added as a silica source. The resulting dispersion was stirred at room temperature for 4 hours. The reaction product was washed with ethanol and was dried to obtain core-shell silica nanoparticles. The yield was 0.18 g.

### Synthesis of Hollow Silica Nanoparticles

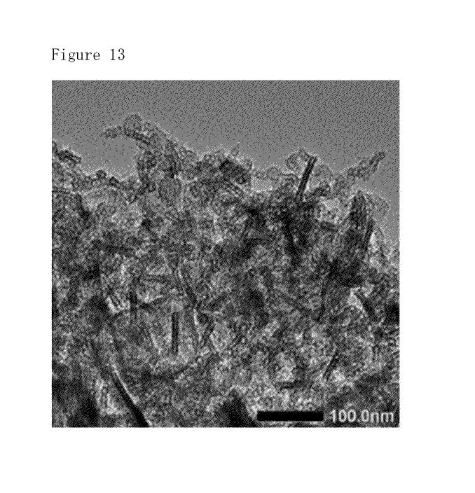

As in Example 1, 0.1 g of the thus-obtained string-shaped core-shell silica nanoparticles were calcined. The yield was 0.07 g. TEM examination confirmed that the resulting silica nanoparticles were string-shaped and had an outer diameter (major axis) of 15 nm and a pore major axis of 4.0 nm (FIG. 13).

1. A monodisperse core-shell silica nanoparticle comprising a core layer based on a hydrophobic organic segment (a2) portion of a copolymer (A) comprising an aliphatic polyamine chain (a1) containing primary amino groups and/or secondary amino groups and the hydrophobic organic segment (a2); and a shell layer comprising a hybrid based on the aliphatic polyamine chain (a1) and silica (B).

2. The core-shell silica nanoparticle according to claim 1, wherein the core-shell silica nanoparticle has an average particle size of 5 to 30 nm.

3. The core-shell silica nanoparticle according to claim 1, further comprising a polysilsesquioxane.

4. A method for manufacturing the core-shell silica nanoparticle according to claim 1, the method comprising the steps of mixing the copolymer (A) comprising the aliphatic polyamine chain (a1) containing primary amino groups and/or secondary amino groups and the hydrophobic organic segment (a2) with an aqueous medium to form an aggregate comprising a core layer based on the hydrophobic organic segment (a2) portion and a shell layer based on the aliphatic polyamine chain (a1); and performing a sol-gel reaction of a silica source using the aggregate as a template.

5. A monodisperse hollow silica nanoparticle having an average particle size of 5 to 30 nm and an inner diameter of 1 to 10 nm.

6. A monodisperse hollow silica nanoparticle formed by removing the copolymer (A) from the core-shell silica nanoparticle according to claim 1, the hollow silica nanoparticle having an average particle size of 5 to 30 nm and an inner diameter of 1 to 10 nm.

7. The hollow silica nanoparticle according to claim 5, further comprising a polysilsesquioxane.

8. A method for manufacturing the hollow silica nanoparticle according to claim 5, the method comprising mixing a copolymer (A) comprising an aliphatic polyamine chain (a1) containing primary amino groups and/or secondary amino groups and a hydrophobic organic segment (a2) with an aqueous medium to form an aggregate comprising a core layer based on the hydrophobic organic segment (a2) and a shell layer based on the aliphatic polyamine chain (a1); performing a sol-gel reaction of a silica source using the aggregate as a template; and removing the copolymer (A).

9. A hollow silica nanoparticle manufactured by the method of manufacture according to claim 8.

10. The core-shell silica nanoparticle according to claim 2, further comprising a polysilsesquioxane.

11. A method for manufacturing the core-shell silica nanoparticle according to claim 2, the method comprising the steps of mixing the copolymer (A) comprising the aliphatic polyamine chain (a1) containing primary amino groups and/or secondary amino groups and the hydrophobic organic segment (a2) with an aqueous medium to form an aggregate comprising a core layer based on the hydrophobic organic segment (a2) portion and a shell layer based on the aliphatic polyamine chain (a1); and performing a sol-gel reaction of a silica source using the aggregate as a template.

12. A method for manufacturing the core-shell silica nanoparticle according to claim 3, the method comprising the steps of mixing the copolymer (A) comprising the aliphatic polyamine chain (a1) containing primary amino groups and/or secondary amino groups and the hydrophobic organic segment (a2) with an aqueous medium to form an aggregate comprising a core layer based on the hydrophobic organic segment (a2) portion and a shell layer based on the aliphatic polyamine chain (a1); and performing a sol-gel reaction of a silica source using the aggregate as a template.

13. A method for manufacturing the core-shell silica nanoparticle according to claim 10, the method comprising the steps of mixing the copolymer (A) comprising the aliphatic polyamine chain (a1) containing primary amino groups and/or secondary amino groups and the hydrophobic organic segment (a2) with an aqueous medium to form an aggregate comprising a core layer based on the hydrophobic organic segment (a2) portion and a shell layer based on the aliphatic polyamine chain (a1); and performing a sol-gel reaction of a silica source using the aggregate as a template.

14. The hollow silica nanoparticle according to claim 6, further comprising a polysilsesquioxane.

15. A method for manufacturing the hollow silica nanoparticle according to claim 6, the method comprising mixing a copolymer (A) comprising an aliphatic polyamine chain (a1) containing primary amino groups and/or secondary amino groups and a hydrophobic organic segment (a2) with an aqueous medium to form an aggregate comprising a core layer based on the hydrophobic organic segment (a2) and a shell layer based on the aliphatic polyamine chain (a1); performing a sol-gel reaction of a silica source using the aggregate as a template; and removing the copolymer (A).

16. A method for manufacturing the hollow silica nanoparticle according to claim 7, the method comprising mixing a copolymer (A) comprising an aliphatic polyamine chain (a1) containing primary amino groups and/or secondary amino groups and a hydrophobic organic segment (a2) with an aqueous medium to form an aggregate comprising a core layer based on the hydrophobic organic segment (a2) and a shell layer based on the aliphatic polyamine chain (a1); performing a sol-gel reaction of a silica source using the aggregate as a template; and removing the copolymer (A).

17. A method for manufacturing the hollow silica nanoparticle according to claim 14, the method comprising mixing a copolymer (A) comprising an aliphatic polyamine chain (a1) containing primary amino groups and/or secondary amino groups and a hydrophobic organic segment (a2) with an aqueous medium to form an aggregate comprising a core layer based on the hydrophobic organic segment (a2) and a shell layer based on the aliphatic polyamine chain (a1); performing a sol-gel reaction of a silica source using the aggregate as a template; and removing the copolymer (A).

18. A hollow silica nanoparticle manufactured by the method of manufacture according to claim 15.

19. A hollow silica nanoparticle manufactured by the method of manufacture according to claim 16.

20. A hollow silica nanoparticle manufactured by the method of manufacture according to claim 17.
